SEQUENCE LISTING (134 sequences)

SEQ ID NO: 1 - DNA, 25 bp, Artificial Sequence
  primer
aagaggtctctgggcgctcacactt

SEQ ID NO: 2 - DNA, 28 bp, Artificial Sequence
  Primer
aagtactgagcatcaggactgtatgggg

SEQ ID NO: 3 - DNA, 25 bp, Artificial Sequence
  Primer
ccacaggtgtgcccatacagacatt

SEQ ID NO: 4 - DNA, 25 bp, Artificial Sequence
  Primer
caggtgaactgggtacgcacactca

SEQ ID NO: 5 - DNA, 30 bp, Artificial Sequence
  Primer
tgtatcagaggcaataatttccaaagcaga

SEQ ID NO: 6 - DNA, 30 bp, Artificial Sequence
  Primer
cgatcccagacactgaagatgaaataagtc

SEQ ID NO: 7 - DNA, 26 bp, Artificial Sequence
  Primer
ggctgttgactgcactgggatttaga

SEQ ID NO: 8 - DNA, 28 bp, Artificial Sequence
  Primer
tcattaactctcagactgacctgggagc

SEQ ID NO: 9 - DNA, 30 bp, Artificial Sequence
  Primer
tatgtgtggcagtgaagagaacaggtcttt

SEQ ID NO: 10 - DNA, 32 bp, Artificial Sequence
  Primer
gggctatcttaagaaatatgaatactttggct

SEQ ID NO: 11 - DNA, 32 bp, Artificial Sequence
  Primer
taatggtaaacaatattttcagccactttgga

SEQ ID NO: 12 - DNA, 28 bp, Artificial Sequence
  Primer
cggatgggtgggtatattttattttcca

SEQ ID NO: 13 - DNA, 28 bp, Artificial Sequence
  Primer
aagagcctctgtttatgtggattgtggc

SEQ ID NO: 14 - DNA, 31 bp, Artificial Sequence
  Primer
tcttggtaaattgccatttttcataaaacaa

SEQ ID NO: 15 - DNA, 26 bp, Artificial Sequence
  Primer
gcttgctatcacggtgtattgggcat

SEQ ID NO: 16 - DNA, 29 bp, Artificial Sequence
  Primer
tgtcagaaggggttagtgctagtgtttga

SEQ ID NO: 17 - DNA, 28 bp, Artificial Sequence
  Primer
aacttgcctgcattgtacatcattccta

SEQ ID NO: 18 - DNA, 28 bp, Artificial Sequence
  Primer
ggggatgttattatttctgaagttggcg

SEQ ID NO: 19 - DNA, 26 bp, Artificial Sequence
  Primer
cattgtggttacattaggggaaggca

SEQ ID NO: 20 - DNA, 24 bp, Artificial Sequence
  Primer
ccccgacagttgtgatgtgttcgt

SEQ ID NO: 21 - DNA, 31 bp, Artificial Sequence
  Primer
tgtttgagtgccttataagttctggttttca

SEQ ID NO: 22 - DNA, 32 bp, Artificial Sequence
  Primer
cagatgtagctttaggcattcttttttcttgt

SEQ ID NO: 23 - DNA, 32 bp, Artificial Sequence
  Primer
atatgttcactggctaaactatgtgtatccca

SEQ ID NO: 24 - DNA, 30 bp, Artificial Sequence
  Primer
cctgtttttgtagagccctcaagttaagaa

SEQ ID NO: 25 - DNA, 30 bp, Artificial Sequence
  Primer
accattgggaatatgttaaagaaattggct

SEQ ID NO: 26 - DNA, 30 bp, Artificial Sequence
  Primer
cagaaaacatctcatttttgaccagctaca

SEQ ID NO: 27 - DNA, 28 bp, Artificial Sequence
  Primer
aaggtatgaggagagcagatgcaaaaag

SEQ ID NO: 28 - DNA, 25 bp, Artificial Sequence
  Primer
gcatgacaaaaatcactgggtggtc

SEQ ID NO: 29 - DNA, 32 bp, Artificial Sequence
  Primer
aatggaaaagtatttggtgttttttgaatgtc

SEQ ID NO: 30 - DNA, 28 bp, Artificial Sequence
  Primer
gcctgacaaaggtgaaactcagttgaaa

SEQ ID NO: 31 - DNA, 26 bp, Artificial Sequence
  Primer
gaaaagctacatcccagtgctgaagg

SEQ ID NO: 32 - DNA, 28 bp, Artificial Sequence
  Primer
atttaaggccaccagattgtgaggaaac

SEQ ID NO: 33 - DNA, 30 bp, Artificial Sequence
  Primer
actttcactaacaagcccttaaaccgaaaa

SEQ ID NO: 34 - DNA, 30 bp, Artificial Sequence
  Primer
tgccccattgtacaccaaagaatgttaata

SEQ ID NO: 35 - DNA, 26 bp, Artificial Sequence
  Primer
atttctttcattaaggctggggaggc

SEQ ID NO: 36 - DNA, 26 bp, Artificial Sequence
  Primer
ggccacctctgaggatctgagcttta

SEQ ID NO: 37 - DNA, 22 bp, Artificial Sequence
  Primer
acttttattggcacaggcattc

SEQ ID NO: 38 - DNA, 25 bp, Artificial Sequence
  Primer
gagttgtttctgacccactgatctc

SEQ ID NO: 39 - DNA, 26 bp, Artificial Sequence
  Primer
tcaaataagagttgtcatatcctgct

SEQ ID NO: 40 - DNA, 22 bp, Artificial Sequence
  Primer
tgaatatgtgtggcagtgaaga

SEQ ID NO: 41 - DNA, 19 bp, Artificial Sequence
  Primer
tgtatggggctggctttag

SEQ ID NO: 42 - DNA, 20 bp, Artificial Sequence
  Primer
gcctctgaagaggtctctgg

SEQ ID NO: 43 - DNA, 21 bp, Artificial Sequence
  Primer
tgtgatcaattccaactgctg

SEQ ID NO: 44 - DNA, 22 bp, Artificial Sequence
  Primer
aaggcttaaaggaaatcacgtc

SEQ ID NO: 45 - DNA, 20 bp, Artificial Sequence
  Primer
ttcttgcaggcatgaagcta

SEQ ID NO: 46 - DNA, 20 bp, Artificial Sequence
  Primer
ctcaacccccttctccatag

SEQ ID NO: 47 - DNA, 19 bp, Artificial Sequence
  Primer
ttttcggatgggtgggtat

SEQ ID NO: 48 - DNA, 23 bp, Artificial Sequence
  Primer
aacaatattttcagccactttgg

SEQ ID NO: 49 - DNA, 23 bp, Artificial Sequence
  Primer
agacaccaaagagcctctgttta

SEQ ID NO: 50 - DNA, 30 bp, Artificial Sequence
  Primer
cttctcactaaattatgtcttggtaaattg

SEQ ID NO: 51 - DNA, 22 bp, Artificial Sequence
  Primer
caatacccagcaaaggatatgg

SEQ ID NO: 52 - DNA, 25 bp, Artificial Sequence
  Primer
gcatctgtacatagtaagcctaccg

SEQ ID NO: 53 - DNA, 20 bp, Artificial Sequence
  Primer
ctttttcaaatgctgcttgc

SEQ ID NO: 54 - DNA, 23 bp, Artificial Sequence
  Primer
cagaaggggttagtgctagtgtt

SEQ ID NO: 55 - DNA, 20 bp, Artificial Sequence
  Primer
gtgccattttgattcccatt

SEQ ID NO: 56 - DNA, 19 bp, Artificial Sequence
  Primer
ggcattccagaaccaaaag

SEQ ID NO: 57 - DNA, 22 bp, Artificial Sequence
  Primer
gacctaaaattgcggtcatttc

SEQ ID NO: 58 - DNA, 21 bp, Artificial Sequence
  Primer
gaaaatgcaggcctttcatca

SEQ ID NO: 59 - DNA, 18 bp, Artificial Sequence
  Primer
agttggcgccagaaatga

SEQ ID NO: 60 - DNA, 24 bp, Artificial Sequence
  Primer
atacaagagtccaaggtagccagt

SEQ ID NO: 61 - DNA, 24 bp, Artificial Sequence
  Primer
ggtaggcatgtagaaatacggttc

SEQ ID NO: 62 - DNA, 23 bp, Artificial Sequence
  Primer
ccaggatagcattcaacagtttg

SEQ ID NO: 63 - DNA, 20 bp, Artificial Sequence
  Primer
ccactcactttcttgcatgg

SEQ ID NO: 64 - DNA, 20 bp, Artificial Sequence
  Primer
cccgacagttgtgatgtgtt

SEQ ID NO: 65 - DNA, 20 bp, Artificial Sequence
  Primer
tgtgttgttgctttgcctct

SEQ ID NO: 66 - DNA, 20 bp, Artificial Sequence
  Primer
cattcccacggttagctgtt

SEQ ID NO: 67 - DNA, 23 bp, Artificial Sequence
  Primer
ttgtgtctgcctgtagttcaatg

SEQ ID NO: 68 - DNA, 26 bp, Artificial Sequence
  Description de la Artificial Sequence armoce
gtgattaaacttgtatttcctgaaca

SEQ ID NO: 69 - DNA, 24 bp, Artificial Sequence
  primer
tcttaggaaaagctacatcccagt

SEQ ID NO: 70 - DNA, 21 bp, Artificial Sequence
  Primer
tttaaggccaccagattgtga

SEQ ID NO: 71 - DNA, 27 bp, Artificial Sequence
  Primer
gctatgttcagaaaatgcatctcactc

SEQ ID NO: 72 - DNA, 21 bp, Artificial Sequence
  Primer
tgatgtgtgacagccaatgaa

SEQ ID NO: 73 - DNA, 22 bp, Artificial Sequence
  Primer
gctagatggtcctgtgtcattg

SEQ ID NO: 74 - DNA, 24 bp, Artificial Sequence
  Primer
gtgattaatgtgaacttccagtgc

SEQ ID NO: 75 - DNA, 20 bp, Artificial Sequence
  Primer
ttgctgtggctctaggttgc

SEQ ID NO: 76 - DNA, 22 bp, Artificial Sequence
  Primer
tcggcagttaatgacagtgatg

SEQ ID NO: 77 - DNA, 22 bp, Artificial Sequence
  Primer
ggggatgacaatgaatatgatg

SEQ ID NO: 78 - DNA, 21 bp, Artificial Sequence
  Primer
tggcataacacttagcaagca

SEQ ID NO: 79 - DNA, 20 bp, Artificial Sequence
  Primer
tcagagacaaggtcgctgct

SEQ ID NO: 80 - DNA, 25 bp, Artificial Sequence
  Primer
tttcttcatagctactccaccactg

SEQ ID NO: 81 - DNA, 25 bp, Artificial Sequence
  Primer
agcttcggagaatctatcaaatagc

SEQ ID NO: 82 - DNA, 20 bp, Artificial Sequence
  Primer
gaacgggtaaaatggcaatg

SEQ ID NO: 83 - DNA, 20 bp, Artificial Sequence
  Primer
aaaccagccagttttcctga

SEQ ID NO: 84 - DNA, 25 bp, Artificial Sequence
  Primer
gtactgggtaggtaatacgctgaga

SEQ ID NO: 85 - DNA, 20 bp, Artificial Sequence
  Primer
gccaattactttgcctctcc

SEQ ID NO: 86 - DNA, 20 bp, Artificial Sequence
  Primer
acgggacaagtctgtttggt

SEQ ID NO: 87 - DNA, 25 bp, Artificial Sequence
  Primer
caaaagatggaggctaatatgttga

SEQ ID NO: 88 - DNA, 24 bp, Artificial Sequence
  Primer
aatagtccattagcaaatccttca

SEQ ID NO: 89 - DNA, 20 bp, Artificial Sequence
  Primer
gcagcttccaactggttctt

SEQ ID NO: 90 - DNA, 20 bp, Artificial Sequence
  Primer
ggctaacccagtgagtccaa

SEQ ID NO: 91 - DNA, 19 bp, Artificial Sequence
  Primer
ctgctgcagattgaaccaa

SEQ ID NO: 92 - DNA, 24 bp, Artificial Sequence
  Primer
aacagcatcgctttagatactagg

SEQ ID NO: 93 - DNA, 19 bp, Artificial Sequence
  Primer
gccctgagcctttcaaatc

SEQ ID NO: 94 - DNA, 22 bp, Artificial Sequence
  Primer
tggaacctcagtcacacctaag

SEQ ID NO: 95 - DNA, 20 bp, Artificial Sequence
  Primer
tgtttcaggactgagcacga

SEQ ID NO: 96 - DNA, 19 bp, Artificial Sequence
  Primer
caggaggtgtggccagttt

SEQ ID NO: 97 - DNA, 19 bp, Artificial Sequence
  Primer
atcctccccactgaacctc

SEQ ID NO: 98 - DNA, 20 bp, Artificial Sequence
  Primer
gcccctcttctgctggttat

SEQ ID NO: 99 - DNA, 18 bp, Artificial Sequence
  Primer
cggggcggacaactaatg

SEQ ID NO: 100 - DNA, 25 bp, Artificial Sequence
  Primer
caaacaggactgaatgaaaacaaca

SEQ ID NO: 101 - DNA, 21 bp, Artificial Sequence
  Primer
agccacttgaattacctggaa

SEQ ID NO: 102 - DNA, 19 bp, Artificial Sequence
  Primer
tcttgggaaatcgcctctc

SEQ ID NO: 103 - DNA, 24 bp, Artificial Sequence
  Primer
cgtaaatggaggttaaatggcttc

SEQ ID NO: 104 - DNA, 19 bp, Artificial Sequence
  Primer
ctccgcacttatgctgcaa

SEQ ID NO: 105 - DNA, 20 bp, Artificial Sequence
  Primer
cccgtttacacctgctgagt

SEQ ID NO: 106 - DNA, 22 bp, Artificial Sequence
  Primer
cctggggagtctaggtaagatg

SEQ ID NO: 107 - DNA, 22 bp, Artificial Sequence
  Primer
agtgagccaaaatggactaagg

SEQ ID NO: 108 - DNA, 20 bp, Artificial Sequence
  Primer
tagggcctgtctgactccaa

SEQ ID NO: 109 - DNA, 19 bp, Artificial Sequence
  Primer
atttccctcccaaccctgt

SEQ ID NO: 110 - DNA, 24 bp, Artificial Sequence
  Primer
gatcaaattgaaggacatgagaga

SEQ ID NO: 111 - DNA, 23 bp, Artificial Sequence
  Primer
ctttctcttcccatcttcacttg

SEQ ID NO: 112 - DNA, 21 bp, Artificial Sequence
  Primer
gcatgcttaaggactgtgaaa

SEQ ID NO: 113 - DNA, 25 bp, Artificial Sequence
  Primer
agaagagtgaacgtattgacatgag

SEQ ID NO: 114 - DNA, 19 bp, Artificial Sequence
  Primer
gcttacgggttttcctcca

SEQ ID NO: 115 - DNA, 25 bp, Artificial Sequence
  Primer
ggaatacagcatactcaaataaagg

SEQ ID NO: 116 - DNA, 21 bp, Artificial Sequence
  Primer
ccctgtgcttaatgtctgcaa

SEQ ID NO: 117 - DNA, 22 bp, Artificial Sequence
  Primer
atgatgtggtacctctgtcacc

SEQ ID NO: 118 - DNA, 22 bp, Artificial Sequence
  Primer
tactcttccaggcactgatagg

SEQ ID NO: 119 - DNA, 20 bp, Artificial Sequence
  Primer
atcacagcggtgaagcaaag

SEQ ID NO: 120 - DNA, 23 bp, Artificial Sequence
  Primer
ctttggtcagtagggatccattt

SEQ ID NO: 121 - DNA, 25 bp, Artificial Sequence
  Primer
tgggtgtctgaaatgttaattgagc

SEQ ID NO: 122 - DNA, 20 bp, Artificial Sequence
  Primer
gggtgagttccagcgtttct

SEQ ID NO: 123 - DNA, 28 bp, Artificial Sequence
  Primer
gatattaaacaagtagcatcagacacaa

SEQ ID NO: 124 - DNA, 22 bp, Artificial Sequence
  Primer
tagtatgcagtggctgttgaga

SEQ ID NO: 125 - DNA, 19 bp, Artificial Sequence
  Primer
agtggctccctgtgtctga

SEQ ID NO: 126 - DNA, 21 bp, Artificial Sequence
  Primer
aatgagcttcgttctttggac

SEQ ID NO: 127 - DNA, 20 bp, Artificial Sequence
  Primer
ctcaacatctggacggagca

SEQ ID NO: 128 - DNA, 27 bp, Artificial Sequence
  Primer
aatggagtgtgtacttgtagagagtga

SEQ ID NO: 129 - DNA, 20 bp, Artificial Sequence
  Primer
acgcctggcctgaaagtatt

SEQ ID NO: 130 - DNA, 20 bp, Artificial Sequence
  Primer
gtctgacatcgccctcgtag

SEQ ID NO: 131 - DNA, 20 bp, Artificial Sequence
  Primer
agaagcagaatggggatgaa

SEQ ID NO: 132 - DNA, 23 bp, Artificial Sequence
  Primer
ggagtaatagattctggcatgtg

SEQ ID NO: 133 - DNA, 20 bp, Artificial Sequence
  Primer
tgaactaaaggtgggcagtg

SEQ ID NO: 134 - DNA, 22 bp, Artificial Sequence
  Primer
caatgaagcatttgacaacgtc

(19) United States
(12) Patent Application Publication
(10) Pub. No.: US 2005/0164229 A1
(43) Pub. Date: Jul. 28, 2005

(54) Methods for in vitro detection of cancers by highlighting allelic imbalances in insertion-deletion markers

(75) Inventors: Bernard Grandchamp, Paris (FR); France Mentre, Paris (FR)

Correspondence Address: MERCHANT & GOULD PC, P.O. BOX 2903, MINNEAPOLIS, MN, 55402-0903, US

(73) Assignee: ASSISTANCE PUBLIQUE-HOPITAUX DE PARIS, Paris (FR)

(21) Appl. No.: 10/925,895

(22) Filed: Aug. 24, 2004

Related U.S. Application Data
(63) Continuation of application No. PCT/FR03/00609, filed on Feb. 25, 2003.

(30) Foreign Application Priority Data
Feb. 25, 2002 (FR) 0202380

(51) Int. Cl. C12Q 1/68; G06F 19/00; G01N 33/48; G01N 33/50

(52) U.S. Cl. 435/6; 702/20

(57) ABSTRACT

The invention relates to a method for detection of tumour cells contained in a biological sample by highlighting allelic imbalances in insertion-deletion chromosome markers, said method using multiple amplification of said markers by thermal-dependent chain reaction with calculation of a overall statistical score for all studied markers and comparison of said score with a fixed normal threshold.

The present invention relates to the medical field, in particular to hospital units and services and to cancer clinics, as well as to medical analytical laboratories. More particularly, the invention relates to non invasive diagnosis and/or prognosis and/or monitoring the efficacy of an anti-cancer treatment.

To this end, the invention employs molecular biological techniques for in vitro analysis of biological samples, associated with a statistical method for processing data derived from that analysis, said combination of molecular biology and statistics allowing the detection of genetic anomalies present in the tumors of patients with cancer.

The invention pertains to a method for detecting tumor cells contained in a biological sample by highlighting allelic imbalances in chromosomal insertion-deletion markers, said method using multiplex amplification of said markers by a heat-dependent chain reaction, using the calculation of an overall statistical score over the set of markers studied, and comparing that score with a fixed threshold of normality.

Epidemiologically, bladder cancers (BC) are very common diseases. BCs are the 5^{th} most common cancer in the Western world, where they are responsible for 3% of all deaths linked to cancer. In the United States, BCs represent the 4^{th} and 8^{th} most common cancer in men and women respectively, with 53000 new cases per annum and with a mortality estimated at 12000 cases per annum (1, 2). In France, in line with all other industrialized countries, the incidence of BC is currently increasing. It rose from 13 to 17.4/100000 per annum between 1980 and 1994 (3). In 1990, the number of deaths in France due to BC was about 7/100000 inhabitants, i.e. 5% of all cancer deaths.

Histologically, 95% of BCs correspond to carcinomas of transitional cells. In France, epidermal carcinomas and adenocarcinomas are still rare.

The UICC (Union Internationale contre le Cancer) classification has defined different stages of invasion of the vesical wall as well as various cytological grades. This classification is summarized in Tables I and II below:

TABLE I

| | Histological stage | | | | | | |
|---|---|---|---|---|---|---|---|
| | carcinoma | | | | | | |
| Invasion | in situ | pTa | pT1 | pT2 | pT3a | pT3b | pT4b |
| urothelium | + | + | + | + | + | + | + |
| chorion | − | − | + | + | + | + | + |
| superficial | − | − | − | + | + | + | + |
| muscle | | | | | | | |
| deep | − | − | − | − | + | + | + |
| muscle | | | | | | | |
| perivesical | − | − | − | − | − | + | + |
| fat | | | | | | | |
| neighboring | − | − | − | − | − | − | + |
| organs | | | | | | | |

TABLE II

| Histological | Nuclear | Mitotic | |
|---|---|---|---|
| grade | atypia | activity | Differentiation |
| G1 | + | + | well differentiated cell |
| G2 | ++ | ++ | medium differentiated cell |
| G3 | +++ | +++ | slightly differentiated cell |

Symptomatically speaking, hematuria and irritative urinary function signs (pollakiuria, urgency, leakage) represent the essential clinical manifestations of BCs.

More rarely, complications linked to lesions are observed (obstructions in the ureter, pelvic compression phenomena) or to its dissemination (distant metastases).

Upon initial diagnosis, about 70% to 80% of tumors are classified as superficial (i.e. do not invade the vesical muscle), and 20% to 30% are invasive. Of the latter, more than half present ganglionic or systemic invasion from the initial diagnosis.

Following trans-urethral resection, which may be associated with a supplemental treatment (immunotherapy or endovesical chemotherapy), the natural history of superficial lesions is marked by two major risks, namely recurrence and progression.

In the light of their importance both on epidemiological and progressive terms, it thus appears necessary to monitor superficial BCs carefully.

Monitoring can be carried out by different methods, the suitability of which depends on the case. Sometimes, they are insufficient by themselves, and are only useful if combined, and in any case, have a certain number of advantages and disadvantages which will be summarized below.

Although rarely contributive, clinical examination is important to the investigation of hematuria (macroscopic or microscopic, determined using urinary dipsticks), and to the appearance of irritative micturitional problems (pollakiuria, urgency). Finally, pelvic examination can detect infiltration of the vesical base, a sign of the infiltrating nature of a tumor.

Cytoscopy is carried out using a rigid cytoscope or a flexible fibroscope. This examination, while effective, is invasive, sometimes poorly tolerated and may be the origin of complications (stenosis of the ureter, urinary infections). Further, cytoscopy does not always allow a proper exploration of the anterior face of the bladder, nor of the intradiverticular regions.

Urinary cytodiagnosis has the twin advantage of being both simple and non invasive. However, its overall sensitivity, i.e. its capacity to detect the anomaly when it is present, is low (between 40% and 60%) (4, 5). This sensitivity is highly dependent on the anatomopathologist making the examination as a substantial part of the final interpretation is subjective in nature. Further, the sensitivity and specificity of urinary cytodiagnosis varies considerably with the tumor grade, specificity as used here designating the aptitude of the method to come to the correct conclusion in the absence of anomalies. In the case of high grade lesions (G3, see Table II above), sensitivity has been determined to be 90% and its specificity, 98% (6). In contrast, the sensitivity drops to between 11% and 25% for low grade tumors (G1, ibid,) (6).

A further method for monitoring BCs consists of assaying Nuclear Matrix Protein 22 (NMP22). The protein is involved in the organization of the mitotic spindle. In fact, it is assayed in urine using a quantitative immuno-enzymatic test. Depending on the positivity limits selected, the sensitivity remains highly variable (7). However, the majority of studies found sensitivities and specificities of 60% to 70% (8). The advantage of this test lies in the fact that its sensitivity does not depend on the tumor grade.

The “BTA Trak Test” uses monoclonal antibodies directed against human complement factor H, the urinary content of which is correlated to the presence of BC. This is a quantitative test with a sensitivity of between 62% and 77%, strongly linked to the grade (G1: 48%; G2: 59%; and G3: 88%) (8, 9). In contrast, it has a lower specificity: 48% to 65% depending on the study. This is due to the fact that the test not only reacts in the case of tumoral lesions, but also with blood factor H in the case of hematuria. Thus, any circumstance which could cause hematuria could be the source of false positives (8).

Hyaluronic acid (HA) is a glycosaminoglycane which is degraded by hyaluronidase (HAase) into small fragments involved in cell adhesion and angiogenesis. In the context of the HA-HAase test, the amounts of these two elements are assayed in urine using an ELISA technique. A sensitivity of the order of 90% (independent of the tumoral grades and stages) and a specificity of 84% have been reported (8, 10).

“Immunocyt” consists of a method combining conventional urinary cytology and an immunofluorescence test, that test using three monoclonal antibodies directed against two mucins and a form of the angiotensin conversion enzyme (ACE). The sensitivity of that combinatorial method is 90% (independent of the tumoral grade) and its specificity is 98% in healthy controls. However, the specificity drops considerably in patients with benign urological infections: 85% in the case of microhematuria, 60% in the case of cystitis and 50% in the case of a prostate adenoma (8, 11).

Initially explored to demonstrate in vitro telomerasic activity (PCR-ELISA kit), the latest versions of the method for assaying said activity are based on quantifying messenger RNA (mRNA) encoding telomerase by RT-PCR (reverse transcription coupled with the polymerase chain reaction). The sensitivity of that technique varies from 0 to 83%; its specificity is of the order of 70% (12, 13). The specificity can drop in the case of an inflammatory process seated in the urinary apparatus, however (expression of telomerase by activated lymphocytes). Further, the existence of macroscopic hematuria can cause the detection of false negatives. Finally, the RT-PCR technique necessitates quasi extemporaneous treatment of urine (12).

It should be noted that a number of other markers that can be used to detect BCs are currently being developed (cytokeratin 20, CD44 variations, BLCA-4 type NMP, uroplakin, . . . ).

However, there is currently no method for surveillance and detection of BCs which is really outstanding as regards its performance in terms of sensitivity and specificity.

In such a context, the determination of genetic anomalies allowing reliable, non invasive detection of BCs would represent a considerable advance.

Tumors are formed by an accumulation of genetic mutations which alter the function of specific genes, namely oncogenes and tumor suppressing genes. Such an accumulation can be accelerated by a major chromosomal instability, which results in a loss of one or more entire chromosomes, or only parts of a chromosome, or even duplication of a chromosome, or uniparental disomy (i.e. the loss of one chromosome then duplication of the remaining chromosome).

In a given individual, a comparison of the nucleic acid from normal tissue (for example leukocytary DNA) and tumoral tissue can allow the determination of a modification or loss of chromosomal material and thus reveal a tumoral process.

An individual who is heterozygous for a polymorphic chromosomal marker has one copy of each allele of said marker. The theoretical ratio between these two alleles, then, is 1 (i.e. 1/1).

Certain genetic anomalies are capable of modifying this ratio. By definition, “allelic imbalances (AI) designate any modification to the ratio between the allelic copies of a marker.

Genetic anomalies can be of different natures. It may be: (i) an increase in the number of chromosomes (for example, trisomy, changing the ratio from 1/1 to 2/1); (ii) deletion to a greater or lesser extent of the chromosomal region containing the allele; (iii) monosomy (change of the ratio from 1/1 to 1/0); or acquired uniparental disomy (change of ratio from 1/1 to 2/0).

Microsatellites are short DNA sequences composed of mono-, di-, tri- or tetranucleotides, said sequences being repeated N times (N being from 10 to 60). About 100000 microsatellite sequences are distributed randomly and homogeneously in the human genome (14). Said sequences are usually located in the non coding regions, although they can also be present in introns or in exons of genes.

Microsatellites are characterized by high individual stability, such that each has its own unique microsatellite allelic distribution. Notwithstanding this stability on the scale of the individual, microsatellites are highly polymorphic between individuals. In this regard, said microsatellites are multi-allelic, and a large number of different alleles, a function of the number of repetitions, can exist. Further, their degree of heterozygosity is from 70% to 95%.

In particular, for BCs, the literature has a large number of genetic anomalies identified as being the origin of AI.

Thus, AIs have been demonstrated in BCs using microsatellite markers since 1994 (15). Said markers have been proposed as a diagnostic tool from 1996 (16). Since then, a number of teams have reported results derived from diagnosis (17; 18; 19) or surveillance (20) BCs using microsatellite markers.

In those studies, the sensitivities were from 84% to 91%, and the specificities were 85% to 100%, using between 10 and 20 microsatellite markers.

However, the use of that type of marker to detect AI has two major disadvantages.

Firstly, from a technical viewpoint, it is very difficult or even impossible to co-amplify a plurality of microsatellite sequences in the same reaction so that each is representative of a given marker. As indicated above, the microsatellite sequences are identically repeated a large number of times and can be common to different markers. This renders simultaneous amplification during a single PCR reaction to amplify a plurality of those markers difficult or even impossible. In practice, the primers cannot be designed to amplify only one copy of a microsatellite sequence without simultaneously amplifying other copies repeated within the same marker, as well as copies of said same sequence belonging to other markers. As a result, each microsatellite marker has to be amplified individually. Since at least ten microsatellite markers have to be studied for a given individual, employing tests using said markers is not only costly as regards time and reagents, but also consumes large quantities of DNA, which may constitute an additional limitation which would be detrimental to its routine use.

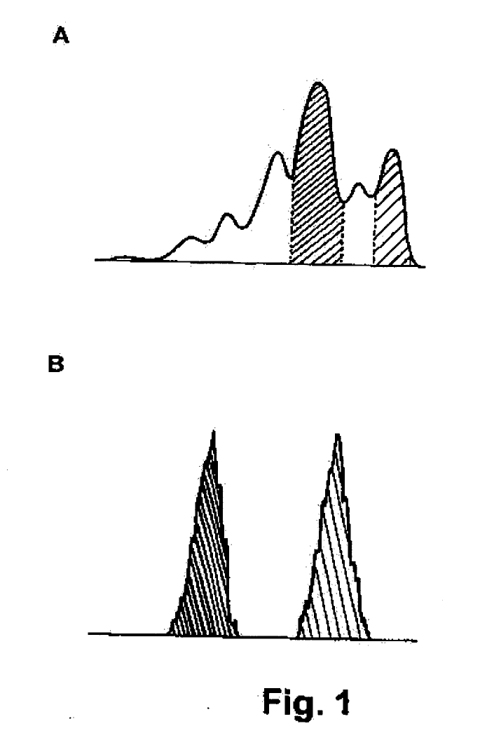

Secondly, the principle of such tests lies in highlighting a modification in the ratio of the surface area of signals corresponding to the amplification products of two alleles of a microsatellite (FIG. 1A). Briefly, DNA is amplified and the ratio of the surface area of the signals in the biological sample to be analyzed, for example of urinary origin when BCs are being studied, and a reference sample, for example from blood, is measured. In order to conclude the existence of an AI, a significant difference between the surface area ratios of the signals relating to the two samples has to be demonstrated. To achieve this, the observed difference must be attributable to experimental fluctuations if appropriate, and in all other cases it can correctly be interpreted as reflecting the presence or absence of an AI. In practice, with microsatellites, the experimenter cannot control amplification biases which are the source of erroneous interpretations, as the reference sample itself can produce a ratio other than 1 (1/1) for a heterozygous individual. Further, the ratios in question vary from one individual to another since, as indicated above, each individual possesses two particular alleles of a microsatellite from a large number of possibilities. Further, the significance limits are only valid for a given individual. Thus, they are selected empirically by the experimenter, as shown by the observed differences in the literature from author to author.

To overcome those major disadvantages, both as regards feasibility and interpretation of the results, to demonstrate AIs, the present invention uses markers other than the usual microsatellites, namely biallelic polymorphic insertion-deletion markers or SIDP, short insertion-deletion polymorphism.

SIDPs are characterized by the presence or absence of several nucleotides. If, for example, we take the following sequence: . . . ATTTGCGCTAAGCTAGCTATTAT . . . , it corresponds to a SIDP if it is capable of containing one or two copies of the short sequence GCTA.

By definition, SIDPs are biallelic, as there are only two different alleles of the same marker. As a result, the individual can be homozygotic for one of the two alleles, or heterozygotic, thus having the two different possible alleles (FIG. 1B).

The degree of heterozygosity of SIDPs is lower than that of microsatellites. In particular, this degree is of the order of 40% for the markers that are used to study BCs (see examples below).

SIDPs have been identified and determined by Doctor Weber's team at the Marshfield (Wis.) “Center for Medical Genetics”. SIDP sequences are available at the following site: http://www.research.marshfieldclinic.org.

Thus, the invention consists of a novel test for detecting tumors, in particular bladder tumors, said test residing in demonstrating the presence of AI in cells contained in a biological sample, for example a urinary sample, using biallelic SIDP type markers.

The present invention provides a method for detecting tumor cells contained in a biological sample by highlighting the presence of AIs in at least fifteen, preferably at least eighteen and more preferably at least thirty to at least forty-eight chromosomal insertion-deletion markers, said method comprising at least the following steps:
a) amplifying said markers by quantitative multiplex PCR;
b) calculating the ratio R of the height of two peaks corresponding to two alleles of each marker;
c) calculating an overall statistical score relating to the set of markers, obtained by comparing the ratio R with a reference ratio Rf; and
d) comparing said score with a fixed threshold of normality.

In the context of the invention, the terms “process”, “method” and “test” are equivalent.

In the context of the present invention, “detection” of tumor cells means demonstrating the presence of such cells. Said “detection” is used for non invasive diagnosis of tumors and/or for prognosis after identifying tumors and/or for surveillance of an anticancer treatment.

The terms “cancer” and “tumor” are equivalent, as is conventional in medical usage.

Again as is conventional in medical usage, a “biological sample” or a “sample” designates any element extracted from a living body, in particular from the human body, such as blood, biopsy, urine, sputum, said element containing cells and being used for diagnostic purposes.

The term “marker” means a particular nucleic acid sequence which can allow specific detection and surveillance of AIs in a sample containing cells. Said marker can be assimilated into an AI “tracer” in said cells. To this end, the marker must be informative, i.e. have as high a degree of heterozygosity as possible, to provide an element of comparison which is capable of being exploited, for example as regards the difference between signals representing the amplification products of two alleles of said marker (FIG. 1B).

With reference to step a) of the above detection method, the term “amplification” is construed in a generic sense, in that it encompasses any molecular biological technique the principle of which consists of amplifying or multiplying a target nucleic acid sequence. Non limiting examples which can be cited are PCR, transcription mediated amplification (TMA), nucleic acid sequence based amplification (NASBA), self-sustained sequence replication (3SR), strand displacement amplification (SDA) and the ligase chain reaction (LCR).

It should be noted that the acronym “PCR” can be used in the context of the present invention to designate the PCR reaction in particular, or any amplification reaction, in particular selected from those cited above.

The amplification reactions envisaged above can be isothermal or necessitate cyclic exposure to different temperatures. The latter configuration is advantageously termed a “heat-dependent” amplification reaction, although this latter term can sometimes be a tacit term, in which case the skilled person is free to deduce it without ambiguity from the context.

According to the usual terminology used in PCR, the sequences of nucleic acids to be amplified are termed “targets” or “matrices”. In the particular context of the present invention, said sequences correspond to markers as defined above and, more particularly, chromosomal markers.

The analysis carried out in the method of the present invention is “quantitative” in that it allows quantification of amplification products corresponding to two alleles of the same marker, and thus provides the basis for calculating the ratio of the peak heights relative to said alleles (step b) of the method), as well as the element to be compared in step c) of said method. As an example, one of the primers for each of the pairs used during step a) can be labeled with a fluorochrome.

Step a) of the method of the invention concerns “multiplex” amplification reactions as long as, rather than an amplification reaction using a unique pair of primers, a plurality of concomitant amplification reactions, preferably 3, of a plurality of different target sequences present in the same sample is carried out, said reactions employing a plurality of distinct pairs of primers, at least ten and preferably at least fifteen pairs of primers.

In accordance with the method of the present invention, the target nucleic acid sequences are amplified using a pair of specific nucleotide primers, the expression “specific primer” designating a nucleotide sequence that can be used as a primer for amplification and is capable of hybridizing under strict conditions with at least 80% of the target nucleic acid sequences. The “strict hybridization conditions” satisfy the conventional definition and are known to the skilled person (21).

The terms and expressions “primers”, “primers for amplification” and “nucleotide primers” are equivalent within the context of the invention. It is understood that the primers are necessarily specific, even if not explicitly mentioned in the text.

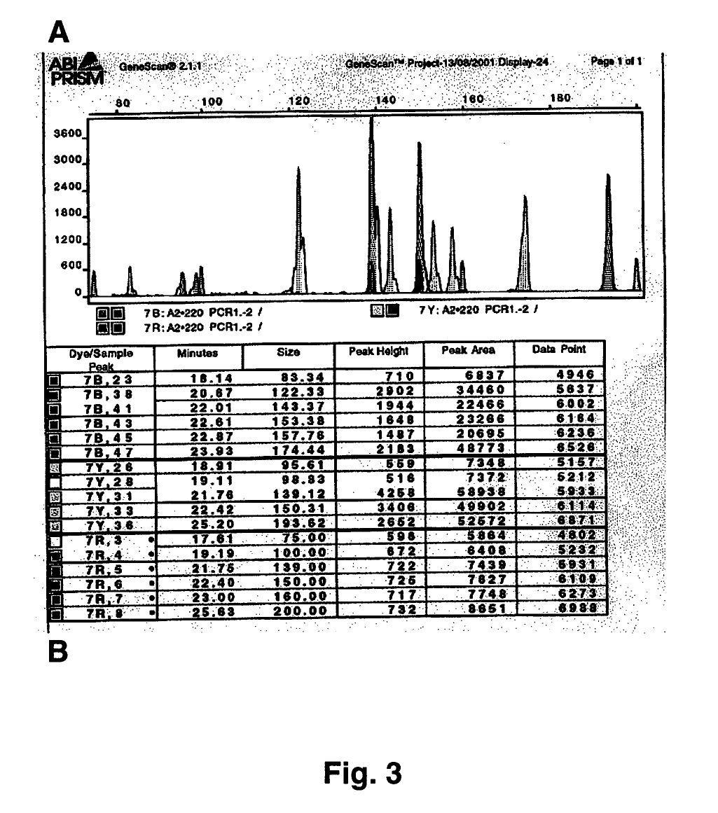

Calculation of the ratio R as it appears in step b) of the method consists of a simple ratio which is accessible to the skilled person provided that a trace is available as shown in FIGS. 1B and 3 and provided by an automatic nucleic acid sequencer.

In practice, a sequence analyzer or automatic sequencer of the ABI PRISMS® (Perkin-Elmer, Wellesley, Mass.) type determines a curve showing the base length of the amplification products along the abscissa and the intensity of the fluorescence emitted by the fluorochrome up the ordinate. The size of the products and the type of fluorochrome associated with said products allows signals relating to the different co-amplified markers to be identified.

As illustrated in FIG. 3, the signals or curves obtained are in the form of peaks of differing heights.

In the case of homozygosity, a single peak is observed. In contrast, in the case of heterozygosity, two peaks corresponding to the two alleles present are separated by a distance proportional to the size of the polymorphism (i.e. the size of the sequence separating the two repeated copies).

In an advantageous implementation of the detection method of the present invention, for a biological sample to be tested, step a) for multiplex PCR amplification is carried out in duplicate; and during step b), rather than calculating the ratio R, the mean of two ratios is calculated, each of said ratios corresponding to an amplification reaction.

In step c) of the method of the invention, reference is made to an “overall statistical score”. Said score represents a standardized statistical entity which allows a threshold of significance to be established (or threshold of normality, or confidence threshold) which is non empirical, fixed and valid for all of the individuals studied. Thus, it is possible to conclude whether a sample is normal or abnormal for all of ratios Rᵢ, calculated for each individual, based on fixed, objective and independent criteria for the individual concerned. The “globalization” or summation of ratios Rᵢ over all of the tested markers for a given individual was dictated by the fact that not all individuals possess the same informative markers, or the same number. Within the context of the invention, this disadvantage is precisely compensated by the quantification of an overall AI corresponding to an individual and no longer to a marker.

In this step, the “reference ratio R_{f}” used to calculate the “overall statistical score” corresponds to a mean of the ratios of the peak heights relative to the two alleles of a marker, said ratios having been obtained from at least twenty, and preferably at least thirty reference samples containing healthy DNA. Said reference samples can derive from different individuals, the only condition which they have to fulfill being to represent healthy samples.

The “reference samples” are biological samples that are carefully selected to be capable of acting as a control in that they represent a biological fluid or a tissue which is healthy as regards the genetic anomalies being investigated. Thus, regarding detecting BCs, the “biological sample” to be tested is of urinary origin, while the “reference samples” will, for example, consist of blood samples.

We then distinguish the “biological sample” to be analyzed, which may contain tumor cells, from the “reference sample”, acting as normality controls, i.e. comprising healthy cells, free of genetic anomalies characteristic of the tumors being investigated.

The “overall statistical score” is obtained using the following formula:
Score=Σdᵢ²=Σ{[LnRᵢ−m(LnR_{f})]÷s(LnR_{f})}²,
in which:
dᵢ represents the statistical distance calculated for a marker i;
Rᵢ designates, for each marker, the ratio of the heights of two peaks, calculated from the biological sample;
R_{f} designates, for each marker, the ratio of the heights of two peaks calculated from a reference sample;
m(LnR_{f}) corresponds to the mean of values of LnR_{f}; and
s(LnR_{f}) corresponds to the standard deviation of values of LnR_{f}.

In this formula, the ratio Rᵢ of the height of the two peaks corresponding to the two alleles of marker i is calculated from the nucleic acid contained in the biological sample and compared with the mean of the reference ratios R_{f} corresponding to the reference samples.

It is understood that steps a) and b) of the detection method are carried out both for the biological sample and for the reference samples, to establish a comparison in accordance with step c) of said method.

To render the representation of ratios Rᵢ and R_{f} symmetrical with respect to 1 (ideal ratio 1/1 for a healthy heterozygotic individual) in the sense that it is immaterial whether the height of the first peak is divided by that of the second or vice versa, provided that the entity represented in the two cases corresponds to the same separation from 1 (for example, ratios 1/2 and 2/1 represent the same separation with respect to 1/1), the variables studied have been transformed into their Napier logarithms.

This transformation shows that the variables follow a Gaussian law, which is well known to statisticians.

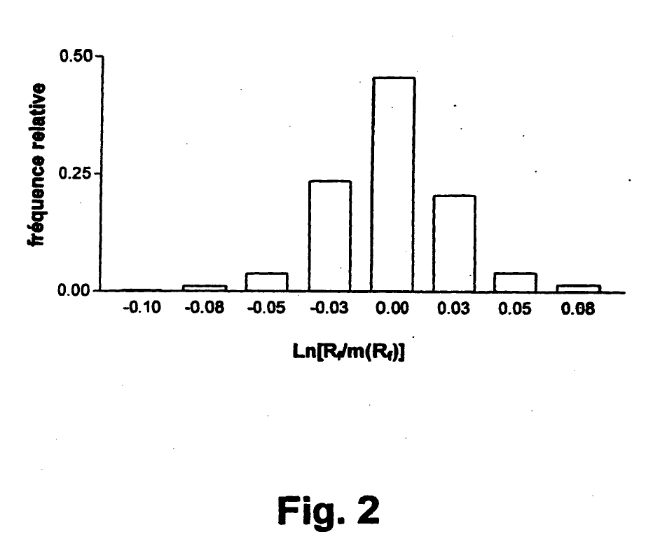

A curve illustrating the Gaussian distribution, termed a “Gaussian” curve or “bell” curve, is shown in FIG. 2 below.

Thus, means and standard deviations characteristic of the Gaussian law verified by the variable LnR_{f} could be calculated. Said means and standard deviations are values estimated from experimental results.

These values are respectively represented by the Latin letters m and s and not by the Greek letters μ and σ, applied to theoretical values for these same statistical entities.

The principle of calculating the overall score lies in that of individual distances for each marker, and summing the squares thereof.

In accordance with the formula above and using routine statistical terminology, a “distance” d corresponds, to a deviation from the mean. In fact, the distance dᵢ represents the distance between LnRᵢ and m(LnR_{f}). Subtracting Gaussian distributions also produces a Gaussian distribution. Said distance is larger if: (i) genetic anomalies are present in the biological sample; and (ii) the ratio between the numbers of tumor cells and healthy cells is higher. To standardize the distance obtained, this is compared with the dispersion of reference measurements for each marker, namely the standard deviation s(LnR_{f}).

The overall score Σdᵢ² given by the above formula, in that it represents the sum of squares of a Gaussian law, is distributed in accordance with a chi-squared law with N degrees of freedom, in which N represents the total number of independent informative markers tested. Regarding statistical use, the denominations “chi-two”, “chi squared” and “χ²” are equivalent.

Definitively speaking, the chi squared law is used to fix a non empirical threshold of significance or threshold of normality.

To this end, the risk of the first kind a is selected to determine the expected specificity of the detection method of the invention. In this context the term “specificity” means the probability that the test is negative if the individuals are healthy, said probability ideally being equal to 1.

Then the chi-squared distribution for this selected value of a and N degrees of freedom provides a limiting value Σd²ₘₐₓ, beyond that which it can be concluded, rigorously and objectively, that the biological sample being studied is pathological, in that it effectively contains tumor cells.

In a further implementation, the detection method of the present invention comprises at least the following sub-steps:
a) amplification by multiplex quantitative PCR of at least fifteen, preferably at least eighteen and more preferably at least thirty to at least forty-five SIDP markers;
b) calculation of the ratio R of the height of two peaks corresponding to the two alleles of each marker;
e) calculation of an individual distance dᵢ for each marker i; and
f) comparison of the value of dᵢ at the selected confidence interval.

The individual distance dᵢ discussed above is given by the following formula:
dᵢ=[LnRᵢ−m(LnR_{f})]÷s(LnR_{f})
in which:
Rᵢ designates, for each marker, the ratio of the heights of two peaks, calculated from the biological sample;
R_{f} designates, for each marker, the ratio of the heights of two peaks calculated from a reference sample;
m(LnR_{f}) corresponds to the mean of values of LnR_{f}; and
s(LnR_{f}) corresponds to the standard deviation for values of LnR_{f}.

In order to provide a comparison in accordance with step f) of the above method, a value is applied to the risk of the first kind a, allowing a normality or confidence interval to be defined for the value of LnRᵢ, except for a. This interval corresponds to (1-α); it is thus defined with the exception of α. As an example, the normality interval can be equal to:
m(LnR_{f})±[3×s(LnR_{f})],
in which case α is 0.1%.

In this example, if an individual distance is obtained which is 3 or more in absolute terms (|dᵢ|≧3), it can be concluded that there is a genetic anomaly in marker i, with an error risk of 0.1% or less.

In general, the detection methods of the invention are applicable to investigating tumors via detection of AI in a given biological sample, provided that informative biallelic insertion-deletion markers are obtained in sufficient number to detect as wide a variety as possible of anomalies which may be encountered in tumor cells the presence of which is being investigated.

In particular, said method can be applied to detecting bladder tumors. In this case, the biological sample is constituted by urine, and the reference sample is blood.

When detecting bladder tumors, at least one marker selected from the markers shown in Table III below is/are used.

TABLE 111

| Location | Marker |
|---|---|
| 2q | 919, 1559 |
| 3p | 17, 752 |
| 4p | 1983 |
| 4q | 642 |
| 5q | 714, 739, 787 |
| 6q | 402, 440, 471, 627 |
| 8p | 12 |
| 9p | 18, 116, 476, 558, 1410, 1570, 1998, 2065, |
| | 2086, 2102 |
| 9q | 118, 289, 475, 516, 561, 682, 1370, 1384, |
| | 1583, 1584, 1654, 1921 |
| 10q | 390, 671, 686, 1789 |
| 11p | 1704 |
| 11q | 1982 |
| 13q | 22, 562, 894, 1363, 1698, 1792, 1979 |
| 14q | 1319 |
| 16q | 792, 1187 |
| 17p | 273, 663, 1385, 1657, 1770 |
| 18q | 1697, 1228 |

In this table, as will be shown in the description below, the term “location” means the chromosomal arm carrying the marker in question.

The markers listed in Table III were selected from SIDP sequences accessible via the website http://www.research/marshfieldclinic.org/, not only because they cover regions carrying genes of interest, namely oncogenes and tumor suppressing genes, but also because they are situated in less targeted zones, and are thus representative of the overall chromosomal instability of BCs.

Regarding amplification of said markers, the method of the invention preferably employs at least one pair of primers constituted by primers for specific amplification, the sequences SEQ ID Nos 1 to 134 of which are described in Table IV below, or sequences hybridizable under strict conditions to at least 80% therewith.

TABLEAU IV

| | | | | | | |
|---|---|---|---|---|---|---|
| | | Seq. | | Seq. | Taille du | |
| | | ID | | ID | produit | Couple |
| Marqueur | Séquence sens | No | Séquence antisens | No | amplifié | d'amorces |

| | | | | | | |
|---|---|---|---|---|---|---|
| 116 | AAGAGGTCTCTGGGCGTCACACACTT | 1 | AAGTACTGAGCATCAGGACTGTATGGGG | 2 | 169/173 | A1 |
| 118 | CCACAGGTGTGCCCATACAGACATT | 3 | CAGGTGAACTGGGTACGCACACTCA | 4 | 138/140 | B1 |
| 17 | TGTATCAGAGGCAATAATTTCCAAAGCAGA | 5 | CGATCCCAGACACTGAAGATGAAATAAGTC | 6 | 136/140 | C1 |
| 18 | GGCTGTTGACTGCACTGGGATTTAGA | 7 | TCATTAACTCTCAGACTGACCTGGGAGC | 8 | 141/144 | D1 |
| 22 | TATGTGTGGCAGTGAAGAGAACAGGTCTTT | 9 | GGGCTATCTTAAGAAATATGAATACTTTGGCT | 10 | 186/192 | E1 |
| 390 | TAATGGTAAACAATATTTTCAGCCACTTTGGA | 11 | CGGATGGGTGGGTATATTTTATTTTCCA | 12 | 171/175 | F1 |
| 402 | AAGAGCCTCTGTTTATGTGGATTGTGGC | 13 | TCTTGGTAAATTGCCATTTTTCATAAAACAA | 14 | 187/191 | G1 |
| 471 | GCTTGCTATCACGGTGTATTGGGCAT | 15 | TGTCAGAAGGGGTTAGTGCTAGTGTTTGA | 16 | 111/114 | H1 |
| 516 | AACTTGCCTGCATTGTACATCATTCCTA | 17 | GGGGATGTTATTATTTCTGAAGTTGGCG | 18 | 97/100 | I1 |
| 561 | CATTGTGGTTACATTAGGGGAAGGCA | 19 | CCCCGACAGTTGTGATGTGTTCGT | 20 | 153/156 | J1 |
| 562 | TGTTTGAGTGCCTTATAAGTTCTGGTTTTCA | 21 | CAGATGTAGCTTTAGGCATTCTTTTTTCTTGT | 22 | 192/194 | K1 |
| 663 | ATATGTTCACTGGCTAAACTATGTGTATCCCA | 23 | CCTGTTTTTGTAGAGCCCTCAAGTTAAGAA | 24 | 126/130 | L1 |
| 671 | ACCATTGGGAATATGTTAAAGAAATTGGCT | 25 | CAGAAAACATCTCATTTTTGACCAGCTACA | 26 | 220/222 | M1 |
| 682 | AAGGTATGAGGAGAGCAGATGCAAAAAG | 27 | GCATGACAAAAATCACTGGGTGGTC | 28 | 193/197 | N1 |
| 686 | AATGGAAAAGTATTTGGTGTTTTTTGAATGTC | 29 | GCCTGACAAAGGTGAAACTCAGTTGAAA | 30 | 210/213 | O1 |
| 714 | GAAAAGCTACATCCCAGTGCTGAAGG | 31 | ATTTAAGGCCACCAGATTGTGAGGAAAC | 32 | 87/89 | P1 |
| 752 | ACTTTCACTAACAAGCCCTTAAACCGAAAA | 33 | TGCCCCATTGTACACCAAAGAATGTTAATA | 34 | 195/197 | Q1 |
| 894 | ATTTCTTTCATTAAGGCTGGGGAGGC | 35 | GGCCACCTCTGAGGATCTGAGCTTTA | 36 | 136/138 | R1 |
| 12 | ACTTTTATTGGCACAGGCATTC | 37 | GAGTTGTTTCTGACCCACTGATCTC | 38 | 129/131 | S1 |
| 22 | TCAAATAAGAGTTGTCATATCCTGCT | 39 | TGAATATGTGTGGCAGTGAAGA | 40 | 128/134 | T1 |
| 116 | TGTATGGGGCTGGCTTTAG | 41 | GCCTCTGAAGAGGTCTCTGG | 42 | 157/161 | U1 |
| 273 | TGTGATCAATTCCAACTGCTG | 43 | AAGGCTTAAAGGAAATCACGTC | 44 | 143/147 | V1 |
| 289 | TTCTTGCAGGCATGAAGCTA | 45 | CTCAACCCCCTTCTCCATAG | 46 | 152/155 | W1 |
| 390 | TTTTCGGATGGGTGGGTAT | 47 | AACAATATTTTCAGCCACTTTGG | 48 | 167/171 | X1 |
| 402 | AGACACCAAAGAGCCTCTGTTTA | 49 | CTTCTCACTAAATTATGTCTTGGTAAATTG | 50 | 208/212 | Y1 |
| 440 | CAATACCCAGCAAAGGATATGG | 51 | GCATCTGTACATAGTAAGCCTACCG | 52 | 105/107 | Z1 |
| 471 | CTTTTTCAAATGCTGCTTGC | 53 | CAGAAGGGGTTAGTGCTAGTGTT | 54 | 122/125 | A2 |
| 475 | GTGCCATTTTGATTCCCATT | 55 | GGCATTCCAGAACCAAAAG | 56 | 215/218 | B2 |
| 476 | GACCTAAAATTGCGGTCATTTC | 57 | GAAAATGCAGGCCTTTCATCA | 58 | 131/134 | C2 |
| 516 | AGTTGGCGCCAGAAATGA | 59 | ATACAAGAGTCCAAGGTAGCCAGT | 60 | 140/143 | D2 |
| 558 | GGTAGGCATGTAGAAATACGGTTC | 61 | CCAGGATAGCATTCAACAGTTTG | 62 | 145/149 | E2 |
| 561 | CCACTCACTTTCTTGCATGG | 63 | CCCGACAGTTGTGATGTGTT | 64 | 122/125 | F2 |
| 627 | TGTGTTGTTGCTTTGCCTCT | 65 | CATTCCCACGGTTAGCTGTT | 66 | 182/187 | G2 |
| 642 | TTGTGTCTGCCTGTAGTTCAATG | 67 | GTGATTAAACTTGTATTTCCTGAACA | 68 | 114/116 | H2 |
| 714 | TCTTAGGAAAAGCTACATCCCAGT | 69 | TTTAAGGCCACCAGATTGTGA | 70 | 92/94 | I2 |
| 739 | GCTATGTTCAGAAAATGCATCTCACTC | 71 | TGATGTGTGACAGCCAATGAA | 72 | 212/216 | J2 |
| 787 | GCTAGATGGTCCTGTGTCATTG | 73 | GTGATTAATGTGAACTTCCAGTGC | 74 | 137/141 | K2 |
| 792 | TTGCTGTGGCTCTAGGTTGC | 75 | TCGGCAGTTAATGACAGTGATG | 76 | 176/180 | L2 |
| 919 | GGGGATGACAATGAATATGATG | 77 | TGGCATAACACTTAGCAAGCA | 78 | 197/200 | M2 |
| 1187 | TCAGAGACAAGGTCGCTGCT | 79 | TTTCTTCATAGCTACTCCACCACTG | 80 | 141/152 | N2 |
| 1228 | AGCTTCGGAGAATCTATCAAATAGC | 81 | GAACGGGTAAAATGGCAATG | 82 | 204/206 | O2 |
| 1319 | AAACCAGCCAGTTTTCCTGA | 83 | GTACTGGGTAGGTAATACGCTGAGA | 84 | 86/99 | P2 |
| 1363 | GCCAATTACTTTGCCTCTCC | 85 | ACGGGACAAGTCTGTTTGGT | 86 | 154/158 | Q2 |
| 1370 | CAAAAGATGGAGGCTAATATGTTGA | 87 | AATAGTCCATTAGCAAATCCTTCA | 88 | 127/129 | R2 |
| 1384 | GCAGCTTCCAACTGGTTCTT | 89 | GGCTAACCCAGTGAGTCCAA | 90 | 200/204 | S2 |
| 1385 | CTGCTGCAGATTGAACCAA | 91 | AACAGCATCGCTTTAGATACTAGG | 92 | 98/116 | T2 |
| 1410 | GCCCTGAGCCTTTCAAATC | 93 | TGGAACCTCAGTCACACCTAAG | 94 | 86/89 | U2 |
| 1559 | TGTTTCAGGACTGAGCACGA | 95 | CAGGAGGTGTGGCCAGTTT | 96 | 158/161 | V2 |
| 1570 | ATCCTCCCCACTGAACCTC | 97 | GCCCCTCTTCTGCTGGTTAT | 98 | 219/224 | W2 |
| 1583 | CGGGGCGGACAACTAATG | 99 | CAAACAGGACTGAATGAAAACAACA | 100 | 185/190 | X2 |
| 1584 | AGCCACTTGAATTACCTGGAA | 101 | TCTTGGGAAATCGCCTCTC | 102 | 104/106 | Y2 |
| 1654 | CGTAAATGGAGGTTAAATGGCTTC | 103 | CTCCGCACTTATGCTGCAA | 104 | 91/95 | Z2 |
| 1657 | CCCGTTTACACCTGCTGAGT | 105 | CCTGGGGAGTCTAGGTAAGATG | 106 | 214/218 | A3 |
| 1697 | AGTGAGCCAAAATGGACTAAGG | 107 | TAGGGCCTGTCTGACTCCAA | 108 | 223/227 | B3 |
| 1698 | ATTTCCCTCCCAACCCTGT | 109 | GATCAAATTGAAGGACATGAGAGA | 110 | 186/188 | C3 |
| 1704 | CTTTCTCTTCCCATCTTCACTTG | 111 | GCATGCTTAAGGACTGTGAAA | 112 | 221/225 | D3 |
| 1770 | AGAAGAGTGAACGTATTGACATGAG | 113 | GCTTACGGGTTTTCCTCCA | 114 | 113/115 | E3 |
| 1789 | GGAATACAGCATACTCAAATAAAGG | 115 | CCCTGTGCTTAATGTCTGCAA | 116 | 188/191 | F3 |
| 1792 | ATGATGTGGTACCTCTGTCACC | 117 | TACTCTTCCAGGCACTGATAGG | 118 | 79/83 | G3 |
| 1921 | ATCACAGCGGTGAAGCAAAG | 119 | CTTTGGTCAGTAGGGATCCATTT | 120 | 110/114 | H3 |
| 1979 | TGGGTGTCTGAAATGTTAATTGAGC | 121 | GGGTGAGTTCCAGCGTTTCT | 122 | 173/177 | I3 |
| 1982 | GATATTAAACAAGTAGCATCAGACACAA | 123 | TAGTATGCAGTGGCTGTTGAGA | 124 | 142/146 | J3 |
| 1983 | AGTGGCTCCCTGTGTCTGA | 125 | AATGAGCTTCGTTCTTTGGAC | 126 | 99/101 | K3 |
| 1998 | CTCAACATCTGCACGGAGCA | 127 | AATGGAGTGTGTACTTGTAGAGAGTGA | 128 | 209/213 | L3 |
| 2065 | ACGCCTGGCCTGAAAGTATT | 129 | GTCTGACATCGCCCTCGTAG | 130 | 164/167 | M3 |
| 2086 | AGAAGCAGAATGGGGATGAA | 131 | GGAGTAATAGATTCTGGCATGTG | 132 | 194/210 | N3 |
| 2102 | TGAACTAAAGGTGGGCAGTG | 133 | CAATGAAGCATTTGACAACGTC | 134 | 115/119 | O3 |

KEY TO TABLE IV:
marker = marker
seq ID No = seq ID No
taille du produit amplifié = size of amplified product
couple d'amorces = primer pair
séquence sens = sense sequence
séquence antisens = antisense sequence

The primers for amplification were selected by applying the following criterion, termed a double specificity criterion, namely a sequence and size specificity.

Firstly, the sequence specificity is such that: (i) said primers can be used simultaneously in the context of multiplex amplification in accordance with the method of the invention, without running the risk of obtaining undesirable secondary amplification products which may falsify the analysis; (ii) they are not mutually hybridizable, which prevents the production of false negatives; and (iii) they are specific for nucleotide sequences which are in turn specific for given markers.

Secondly, the size specificity of the amplification products generated allows unambiguous attribution of each of said products to the marker from which it derives using conventional analytical methods which are known to the skilled person, such as gel electrophoresis.

This double specificity is essential and has been imposed by the technical conditions of the method of the invention. If suitably selected, the primers for amplification are all capable of hybridizing specifically and selectively at similar hybridization temperatures, to obtain amplification products the size of which varies within a relatively narrow interval, for example between 80 and 200 base pairs (bp), to facilitate carrying out the subsequent steps in the detection method.

In a particular implementation, the multiplex quantitative amplification step a) of the detection method, in particular for BCs, comprises at least the following sub-steps:
a1) an initial denaturing step at 95° C. for 7 min;
a2) a denaturing step at 95° C. for 30 seconds;
a3) a hybridization step at 61° C. for 30 seconds;
a4) an elongation step at 72° C. for 30 seconds;
steps a2) to a4) constitute a cycle, said cycle being repeated 35 times; and
a5) a final elongation step at 72° C. for 10 minutes.

These sub-steps are preferably but not exclusively carried out when the amplification primers described above are used. Said sub-steps can also be applied during multiplex SIDP amplification, using primers distinct from those cited in the Table, provided that the criteria mentioned above regarding the hybridization temperatures for said primers are satisfied.

The present invention also envisages sequences SEQ ID Nos 1 to 134 for use as amplification primers (Table IV, above), and sequences hybridizable under strict conditions to at least 80% therewith (21).

Further, the invention concerns pairs of primers AI to O3 identified in Table IV above, and the group they constitute, said pairs, designated AI to O3 consisting of a sense sequence and an antisense sequence selected in pairs from sequences SEQ ID Nos 1 to 134, and sequences hybridizable under strict conditions to at least 80% therewith.

It is clear that designations AI to O3 for the pairs of primers for amplification of the invention comprise sequences SEQ ID Nos 1 to 134 (Table IV, above) and sequences hybridizable under strict conditions to at least 80% therewith.

Finally, the present invention pertains to a kit or system for detecting tumor cells contained in a biological sample by highlighting allelic imbalances in chromosomal biallelic insertion-deletion markers, said kit comprising at least one pair of amplification primers constituted by primers selected in pairs from sequences SEQ ID Nos 1 to 134 and sequences hybridizable under strict conditions to at least 80% therewith, the primer pairs in question being designated AI to O3 in Table IV above.

The present invention will now be illustrated with reference to the following figures which in no way limit the invention:

FIG. 1: An example of the configuration of the results of a test for detecting genetic anomalies in a biological sample from a heterozygotic individual after processing with a nucleic acid sequence analyzer, said nucleic acids being labeled using a fluorochrome. The hatched portions represent the areas of signals corresponding to two alleles of a marker;

FIG. 1A: microsatellites;

FIG. 1B: SIDP.

FIG. 2: Histogram representing variables LnR_{f} for the set of biallelic insertion-deletion markers studied in the context of detecting BCs. In fact, it is the variable Ln[R_{f}/m(R_{f})] which is shown here in order to remove the bias linked to amplification by PCR. The distribution of Ln[R_{f}/m(R_{f})] obeys a Gaussian law;

FIG. 3: An example of a trace provided by GeneScan® 2.1.1 (ABI PRISM@) software when using the detection method of the invention.

FIG. 3A: A trace using nine SIDP markers, four labeled with HEX and five labeled with FAM fluorochromes, as well as a size standard. The size of the amplification products is shown along the abscissa, and the fluorescence intensity is up the ordinate. In this example, only two markers are heterozygotic and gave rise to the detection of two peaks;

FIG. 3B: for each peak, the Table indicates its migration time to the reading window, its size as the number of bases, its height and its area.

The invention will be better understood from the following examples which are given purely by way of illustration. It should be understood that the present invention is not in any way limited to these examples.

### EXAMPLES

The method of the invention for detecting genetic anomalies presented by tumor cells was applied to investigating BCs.

It was elected to test chromosomal regions that are frequently described in the literature as presenting IAs at the origin of BC, in order to have available elements for comparison. Thus, the following chromosomal arms were studied: 9p, 9q, 17p, 10q, 13q, 6q, 3p and 5q.

I—Materials and Methods

I-1—Patients

The criterion for inclusion in the study was the discovery of a BC in persons presenting no antecedents for urogenital cancer.

The controls were patients hospitalized for various urological reasons (stones, urinary incontinence, prostate adenoma, etc).

Only patients and controls having urogenital cancer antecedents were excluded.

87 patients were selected, divided into 24 controls and 63 cases. The histological characteristics of the cases are summarized in Table V below.

TABLE V

| | pTa | pT1 | pT2-4 | Total |
|---|---|---|---|---|
| G1 | 9 | 2 | 0 | 11 |
| G2 | 18 | 6 | 3 | 27 |
| G3 | 1 | 16 | 8 | 25 |
| Total | 28 | 24 | 11 | 63 |

I-2—Markers

The markers were SIDPs the sequences for which are accessible via the website: http://www.research.marshfieldclinic.org.

I-2-1) Number of Markers Studied

Since the degree of heterozygosity, and thus informativity, of microsatellites is higher than that of SIPDs, a larger number of SIDP markers had to be used to produce the same level of informativity.

For this reason, several markers were selected on each chromosomal arm studied. The 9q arm, in that it is the principal seat of anomalies in BC, was studied in particular detail, with four markers.

I-2-2) Selection of Loci

Ideally, and as indicated above, the markers should not only cover regions carrying oncogenes and tumor suppressor genes, but also less specific zones which may present an AI because of the overall chromosomal instability of BCs.

The choice of regions covered by the markers was guided by the literature (Table III, above).

I-3-Quantification of DNA Solutions

For each patient, urinary and leukocytary DNA were extracted from cells contained in the biological and reference sample respectively using methods known to the skilled person.

The DNA solutions, initially diluted to 1/100, were labeled with PicoGreen® fluorochrome which selectively binds to double stranded DNA. The fluorescence was read at 520 nm by comparison with a calibration curve on a fluorimeter. Each sample was then diluted to obtain a DNA concentration of 100 ng/μl. For certain urinary samples, the concentration was less than 100 ng/μl. The final solutions were stored at −20° C.

I-4-PCR Amplification

The first step consisted of amplification of different markers using three multiplex PCR reactions, each amplifying six markers.

I-4-1) DNA

25 ng of DNA was used for PCR. The reactions were carried out with 25 ng of DNA to avoid any limitations due to the quantity of DNA extracted from the urinary sediment.

I-4-2) Primers

Each pair of primers for amplification encompassed an insertion-deletion polymorphism and were labeled on one primer only using a FAM or HEX fluorochrome.

I-4-3) Reaction Medium

It contained a polymerase (Taq Polymerase Gold from Perkin-Elmer, 5 U/μl), a buffer (Buffer 10×, Perkin-Elmer), MgCl₂ (Perkin-Elmer, 25 mM), deoxynucleotide triphosphates (dATP, dTTP, dGTP and dCTP, each 5 mM) and water to obtain a final volume of 25 μl.

VII and VIII below indicate the composition of the reaction media as a function of the primer pairs.

TABLE VI

| COMPONENT | TITER | QUANTITY (μl) |
|---|---|---|
| MgCl₂ | 25 mM | 2.5 |
| Buffer | 10× | 2 |
| dNTP | 5 mM × 4 | 1 |
| Taq | 5 U/μl | 0.25 |
| 516 | 10 pmol/μl | 0.9 |
| 116 | 10 pmol/μl | 1.5 |
| 118 | 10 pmol/μl | 1.5 |
| 18 | 10 pmol/μl | 1 |
| 561 | 20 pmol/μl | 1.75 |
| 682 | 50 pmol/μl | 1 |
| DNA | 50 ng | cf [DNA] |

| | |
|---|---|
| H₂O | complement to 25 μl |

TABLE VII

| COMPONENT | TITER | QUANTITY (μl) |
|---|---|---|
| MgCl₂ | 25 mM | 4 |
| Buffer | 10× | 2.5 |
| dNTP | 5 mM × 4 | 1 |
| Taq | 5 U/μl | 0.25 |
| 894 | 10 pmol/μl | 0.4 |
| 663 | 10 pmol/μl | 2 |
| 402 | 10 pmol/μl | 2 |
| 22 | 10 pmol/μl | 2 |
| 562 | 10 pmol/μl | 4 |
| DNA | 50 ng | cf [DNA] |

| | |
|---|---|
| H₂O | complement to 25 μl |

TABLE VIII

| COMPONENT | TITER | QUANTITY (μl) |
|---|---|---|
| MgCl₂ | 25 mM | 3 |
| Buffer | 10× | 2.5 |
| dNTP | 5 mM × 4 | 1 |
| Taq | 5 U/μl | 0.25 |
| 714 | 10 pmol/μl | 0.3 |
| 471 | 10 pmol/μl | 0.4 |
| 17 | 10 pmol/μl | 0.8 |
| 390 | 10 pmol/μl | 1.6 |
| 752 | 10 pmol/μl | 1.8 |
| 686 | 20 pmol/μl | 2 |
| 671 | 20 pmol/μl | 2 |
| DNA | 50 ng | cf [DNA] |

| | |
|---|---|
| H₂O | complement to 25 μl |

I-4-4) PCR Program

Identical for the three reactions, comprising the following steps:
1 a phase for denaturing and activating polymerase at 95° C. for 7 minutes;
2 a phase for denaturing at 95° C. for 30 seconds;
3 a hybridization phase at 61° C. for 30 seconds;
4 an elongation phase at 72° C. for 30 seconds;
phases 2-3-4 were repeated 35 times;
5 a final elongation of 10 minutes at 72° C.

I-4-5 Carrying Out Multiplex PCR

Each pair of primers was initially checked individually to ensure its reactivity. The primers were then combined in the same reaction mixture.

Multiple runs were then produced by adjusted various parameters influencing the reaction (hybridization temperature, concentration of MgCL₂, polymerase concentration, number of amplification cycles), to determine the optimum amplification conditions.

Further, a major difficulty was linked to the fact that amplification of the different products must be carried out in a relatively equivalent manner so that the conditions for reading the sequence analyzer remained valid regardless of the product (i.e. all amplification products are detected without any saturation).

I-5-Analysis of Amplified Fragments

The amplification products were separated as a function of their size then quantified on an ABI PRISM® analyzer (model 310, 1 capillary, or 3100, 16 capillaries).

This apparatus functioned as follows. The amplification products, labeled with the fluorescent primer, were mixed with a denaturing buffer (formamide) and an internal size standard. This mixture was then denatured (heating 3 min at 95° C.) and deposited in the apparatus. The deposit was charged electrically for 5 sec at 15 kV, then had migrated in a capillary subjected to a large electrical field (15 kV). The capillary was irradiated at a window by a laser beam. The light emitted by the fluorescent molecule due to impact of the laser beam was captured by a CDD digital camera then analyzed using GeneScan® software. The quantity of product present in the deposit was proportional to the intensity of the emitted radiation. Its size was calculated from the migration time of the product during capillary electrophoresis to the window for the laser beam.

FIG. 3 shows an example of a trace.

I-6—Determination of Allelic Imbalance

In theory, an AI results in modification of the 1/1 ratio of the two alleles initially present in the cell (either disappearance or duplication of an allele).

One difficulty was caused by the fact that the urinary sediment contained tumor cells, but also normal cells deriving from desquamation of healthy urothelium. The respective proportions of these two cell populations varied. Under these conditions, an AI is manifested by modifications which are relatively less sensitive to the ratio of the peak heights relative to the two alleles. The extent of these modifications was proportional to the percentage of cells of tumoral origin in the urinary sediment and also depended on the type of anomalies responsible for the AI.

Thus, it was necessary to define criteria enabling a decision to be taken as to whether a modification to the ratio was significant and would effectively correspond to an AI, or whether it concerned an insignificant difference linked to experimental variations.

Statistical calculations were made and are reported in the “II-Results” section below. The results were processed, and the calculations were carried out using ACCESS® software (Microsoft).

II —Results

II-1—Method for Calculating Allelic Imbalances

Two different calculation methods were used:

II-1-1) Definition of Criteria for Each Marker

Determining an AI was based on comparing the ratios R_{f} and Rᵢ of the height of two peaks corresponding to two alleles of the marker under consideration, in the blood and urine respectively.

The ratios employed here were rendered symmetrical with respect to 1 by using Napier logarithms.

For each marker, the variables LnRf were shown to obey a Gaussian law. As a result, an experimental mean and a standard deviation for the LnR_{f}, respectively termed m(LnR_{f}) and s(LnR_{f}), were determined for each marker.

Thus, by selecting an error risk of the first kind a of 0.1%, a normality interval was defined for the value LnRᵢ as being: LnR_{f}±[3×s(LnR_{f})].

Since LnR_{f} is an experimentally measured random individual variable, the mean m(LnR_{f}) is preferred for use in the calculations, to limit experimental fluctuations.

The definition of the 99.9% normality interval was thus:
LnRᵢ in the range [m(LnR_{f})±3×s(LnR_{f})]

Table LX below indicates, for each marker, the amount of informative DNA (i.e. heterozygotic DNA) which served to determine the distribution of LnR_{f}, m(LnR_{f}) and s(LnR_{f}).

TABLE IX

| | Amount of | | |
|---|---|---|---|
| Marker | informative DNA | m(LnR_{f}) | s(LnR_{f}) |
| 17 | 34 | 0.0483 | 0.0193 |
| 18 | 38 | 0.0000 | 0.0521 |
| 22 | 55 | 0.0572 | 0.0351 |
| 116 | 39 | 0.1518 | 0.0347 |
| 118 | 39 | −0.1009 | 0.0281 |
| 390 | 44 | 0.0668 | 0.0189 |
| 402 | 53 | 0.0742 | 0.0200 |
| 471 | 46 | 0.0418 | 0.0563 |
| 516 | 49 | 0.0460 | 0.0188 |
| 561 | 40 | −0.0412 | 0.0257 |
| 562 | 42 | 0.0387 | 0.0355 |
| 663 | 43 | 0.0085 | 0.0212 |
| 671 | 36 | 0.0088 | 0.0130 |
| 682 | 22 | 0.0767 | 0.0220 |
| 686 | 30 | −0.0289 | 0.0268 |
| 714 | 58 | −0.0106 | 0.0172 |
| 752 | 41 | 0.0217 | 0.0150 |
| 894 | 35 | −0.0093 | 0.0184 |

A can be seen from Table IX, m(LnR_{f}) does not always equal 0 (corresponding to m(R_{f})=1), even though there was no AI. This was due to amplification bias, i.e. preferential amplification of one allele rather than the other during the PCR reaction.

This method for determining AIs had the disadvantage of being based on in an accumulation of risks of the first kind a. In fact, although the risk a was fixed for one informative marker, it increased with the number of informative markers. In order to overcome this disadvantage, a risk a correlated with the number of informative markers had to be determined, so that the cumulative risk a was identical from one patient to another.

To this end, the calculation of a unique score taking into account all of the tested markers for a patient was developed.

II-1-2) Calculation of an Overall Score

The principle was based on calculating the distance of Rᵢ from mean values of R_{f} for each marker to produce a sum of squares after logarithmic transformation and to test the score obtained using a chi squared law.

Here again, because of experimental fluctuations, the heights of the peaks from which Rᵢ and R_{f} were calculated could be considered to be random variables the experimental measurements from a PCR reaction of which were estimates.

For a given experimental result Rᵢ, the square of the distance from LnRᵢ to the mean m(LnR_{f}) could be calculated referred to a standard deviation s(LnR_{f}):
dᵢ²={[LnRᵢ−m(LnR_{f})]/s(LnR_{f})}²

For N informative markers, the sum of the squares of the distances was determined, termed the overall score Σdᵢ². Said score is distributed in accordance with a chi squared law with N degrees of freedom. The probability that Σdᵢ² was greater than a given value could thus readily be determined using charts.

An upper limit to Σdᵢ² was fixed, corresponding to a given risk α, determining the specificity of the test for N informative markers. As an example, by selecting a to be 5%, the chi squared distribution for N degrees of freedom allowed Σdᵢ²ₘₐₓ to be fixed such that when, for a given urine sample, a Σdᵢ² was obtained that is above Σdᵢ²ₘₐₓ, it could be objectively concluded that the urine under consideration is pathological, with an error risk of 5%.

II-2-Test Results

II-2-1) Overall Results

To characterize the “performance” of the detection method of the invention as used in cases of BC, “sensitivity” and “specificity” parameters were defined as follows.

“Sensitivity” corresponds to the probability of the test being positive in the case of an anomaly. Ideally, that probability is 1.

“Specificity” as defined above designates the probability that the test is negative in the absence of anomalies. Here again, the ideal probability is 1.

For the calculation per marker, a confidence interval of 0.1% was defined. In the case of AI on one or more markers, the test was considered to be positive. This calculation method provided the following results: a sensitivity of 76.19% (48 out of 63 patients having at least one AI) for a specificity of 95% (1 control out of 17 having at least one AI).

Determination of AI by score revealed a sensitivity of 68.25% (43 out of 63 patients having at least one AI) for a specificity of 95% (1 control out of 17 having at least one AI).

II-2-2) Results Compared with Tumor Stage and Grade

Table X below shows the sensitivities as a function of the calculation method, stage and histological grade.

TABLE X

| | | % sensitivity | % sensitivity |
|---|---|---|---|
| | | (calculation | (calculation |
| | n | by score) | by marker) |
| Overall | 63 | 68.25 | 76.19 |
| pTa | 28 | 57.14 | 67.86 |
| pT1 | 24 | 81.82 | 86.37 |
| pT2-4 | 11 | 72.73 | 81.82 |
| G1 | 11 | 54.55 | 63.64 |
| G2 | 27 | 66.67 | 74.07 |
| G3 | 25 | 78.26 | 86.96 |

This analysis demonstrated a lower sensitivity for pTa lesions with respect to pT1(p=0.02). The sample of pT2-4 tumors was too small to allow a comparison with pT1 using the chi squared test.

In the same manner, the test was less sensitive for G1 lesions than for G2 lesions (P=0.001). There was no significant difference in terms of sensitivity between G2 and G3 tumors (p>0.05).

II-3-Importance of Different Chromosomal Arms

II-3-1) Overall Sensitivity of Chromosomal Arms

Table XI below shows the sensitivity observed for each chromosomal arm.

TABLE XI

| | Arm | | | | | | | |
|---|---|---|---|---|---|---|---|---|
| | 10q | 13q | 3p | 5q | 6q | 9p | 9q | 17p |
| sensitivity | 45% | 40% | 34% | 37% | 41% | 42% | 58% | 33% |

II-3-2) Sensitivity as a Function of Arms and Histological Criteria

Table XII belows the sensitivities of each arm as a function of the tumor stage and grade.

TABLE XII

| | n | 10q | 13q | 3p | 17p | 5q | 6q | 9p | 9q |
|---|---|---|---|---|---|---|---|---|---|
| pTa | 28 | 33% | 19% | 18% | 8% | 16% | 19% | 47% | 48% |
| pT1 | 24 | 53% | 61% | 38% | 44% | 47% | 58% | 50% | 75% |
| pT2-4 | 11 | 50% | 56% | 29% | 50% | 100% | 40% | 0% | 50% |
| G1 | 11 | 20% | 9% | 0% | 0% | 29% | 38% | 38% | 50% |
| G2 | 27 | 41% | 39% | 25% | 15% | 12% | 25% | 50% | 59% |
| G3 | 25 | 57% | 58% | 42% | 50% | 71% | 52% | 38% | 60% |

II-4—Importance of Different Markers

The sensitivity was calculated for each marker. The results are shown in Table XIII below.

TABLE XIII

| Marker | Sensitivity (%) |
|---|---|
| 17 | 10.53 |
| 18 | 29.63 |
| 22 | 25 |
| 116 | 44 |
| 118 | 44.44 |
| 390 | 40 |
| 402 | 51.61 |
| 471 | 20 |
| 516 | 54.29 |
| 561 | 65.52 |
| 562 | 1923 |
| 663 | 27.59 |
| 671 | 41.67 |
| 682 | 31.25 |
| 686 | 36.36 |
| 714 | 39.02 |
| 752 | 37.04 |
| 894 | 48.15 |

III—Conclusions

III-1—Specificity of Test

The specificity of the test can be selected as a function of the value attributed to the risk a (95% for calculation by score in the experiments summarized above, i.e. 1 false positive for 17 controls).

The specificity can be increased by improving the reproducibility of the PCR amplification reactions. In this regard, it is advantageous to carry out each PCR in duplicate. In the case of disagreement of the results, a third discriminating PCR can then be carried out.

III-2—Sensitivity of Test

In accordance with the AI determination method, the test sensitivity was 68.25% (overall score) or 76.19% (individual criteria for AI).

One of these sensitivity limits is based on the number of informative markers.

Initially, this test was designed for thirty markers for which the mean degree of informativity was 42.62%. Said markers were distributed over nine chromosomal arms. Seven of them were explored by three markers each, which represented a mean probability of having at least one informative marker on the arm of 81%. The other two arms were respectively explored by seven markers (arm 9p) and two markers (arm 11p), with probabilities of having at least one informative marker of 99.98% and 68% respectively.

During the first development of the test, only eighteen markers were used, distributed over eight chromosomal arms and having a mean degree of informativity of 43%.

The probability that at least one marker was informative over each chromosomal arm being studied was a mean of 69.5%.

At the end of development of the test, fifty-nine markers were used, distributed over seventeen chromosomal arms.

The sensitivity was improved by using additional markers.

III-3-Advantages of the Test of the Invention

Because of the biallelic nature of the markers used, the amplification products all had the same size (size given ± polymorphism size), and the same appearance, with the exception of microsatellites, which gave rise to the formation of multiple amplification products distinguished from each other by the number of repetitions (FIG. 1, A and B). In the test of the invention, this results in ease of reading and interpretation, which is not the case with microsatellites.

For the same reasons, it has proved possible to use multiplex amplification reaction protocols, which do not function with microsatellites beyond two, or three markers as an absolute maximum. This multiplex operation can compensate for the degree of informativity of SIPDs, which is lower than that of microsatellites, by increasing the number of markers without rendering its production too expensive as regards time, reagents and samples.

Finally, observation of the same profile in all healthy heterozygotic individuals allowed standards to be determined, for each marker and overall, in order to provide a statistically rigorous, objective determination of the presence or absence of AI.

### III-4—Conclusions

AIs have the advantage of being an anomaly which is specific to tumor cells, compared with other known markers (telomerases, fibrinogen degradation factors, factor H protein, etc . . . ).

Following optimization of the test, in particular increasing the number of markers, the use of SIDP type markers results in a specificity of about 95% and a sensitivity of about 80% to 90%.

In conclusion, the tumor detection test, in particular for detecting BCs, of the invention is simple, technically reliable and cheap, and can readily be integrated into the routine activities of a molecular biology laboratory.

This non invasive test can in particular be used in detecting urothelial tumors of the upper urinary apparatus, which are more difficult to detect than BCs.

### REFERENCES

(1) Greenlee R. T., Murray T” Bolden S., et al: Cancer statistics, 2000. CA Cancer J. clin.; 50: 7-33.
(2) Boring C., Squires T., Tong T.: Cancer J. clin.; 1995; 45: 8.
(3) Pfister P., Asselain B., Blanchon B., et al: Evolution de I'incidence des cancers declares a I'assurance maladie en Ile-de-France entre 1980 et 1994, BEH n° 3, 2000.
(4) Grossman H. B.: New methods for detection of bladder cancer. Semin. Urol. Oncol.; 1998; 16: 17-22.
(5) Ramakumar S, Bhuiyan J., Besse J. A., and al.: Comparison of screening methods in the detection of bladder cancer. J. urol.; 1999; 161: 388-394.
(6) Brown F. M.: Urine cytology, is it still the gold standard for screening? Urol. Clin. of North Am.; February 2000; 27(1): 25-37.
(7) Grocela J. A., McDougal W. S.: Utility of nuclear matrix protein in the detection of bladder recurrent cancer. Urol. Clin. of North Am.; 2000 February; 27: 47-51.
(8) Lokeshwar V. B., Soloway M. S.: Current bladder tumor tests: does their projected utility fulfill clinical necessity? J. of urology; April 2001; 165 1 067-1077.
(9) Malkowicz S. B.: The application of human complement factor H related protein (BT A TRAK) in monitoring patients with bladder cancer. Urol. Clin. of north Am.; 2000 February; 27: 63-73.
(10) Lokeshwar V. B., Block N. L.: HA-Haase urine test: a sensitive and specific method for detecting bladder cancer and evaluating its grade. Urol. Clin. of North Am.; 2000 February; 27: 53-61.
(11) Fradet Y., Lockhart C. and al: Performance characteristics of a new monoclonal antibody test for bladder cancer: Immunocyt. The Canadian Journal of Urology; September 1997; 4(3); 400-405.
(12) Bernues M., Casadevall C., Caballin M. R. et al.: Study of allelic losses on 3p, 6q, and 17p in human urothelial cancer. Cancer Genet Cytogenet; 1999 Jul. 1; 112(1): 42-5.
(13) Le Duc A., Cussenot O., Desgrandchamps F.: Les tumeurs. de vessie, ed. Sanofi-Winthrop, 1994.
(14) Uchida T., Wang C., Wada C. et al.: Microsatellite instability in transitional cell carcinoma of the urinary tract and its relationship to clinicopathological variables and smoking. Int J Cancer 69: 142-145, 1996.
(15) Mao L., Lee D. J., Tockman M. S. et al: Microsatellite alterations as clonal markers for the detection of human cancer. Proc. Natl. Acad. Sci. USA; 1994; 91: 9871-9875.
(16) Mao L., Schoenberg M. P., Scicchitano M. and al.: Molecular detection of primary bladder cancer by microsatellite analysis. Science; 1996 Feb. 2; 271(5249): 659-62.
(17) Baron A., Mastroeni F., Moore P. S. and al.: Detection of bladder cancer by semi automated microsatellite analysis of urine sediment. Adv. Clin. Path.; 2000 January; 4(1): 19-24.
(18) Christensen M., Wolf H., Orntoft T.: Microsatellite alterations in urinary sediments from patients with cystitis and bladder cancer. Int J. Cancer; 2000; 85: 614-617.
(19) Schneider A., Borgnat S., Lang H. and al.: Evaluation of microsatellite analysis in urine sediment for diagnosis of bladder cancer. Cancer Res.; 2000 Aug. 15; 60(16): 4617-22.
(20) Steiner G., Schoenberg M. P., Linn J.-F. et al: Detection of bladder recurrence by microsatellite analysis of urine. Nat. Med.; 1997 June; 3(6): 621-4.
(21) Sambrook and Russel. 2001. Molecular cloning: a laboratory manual, 3rd ed., Cold Spring Harbor Laboratory Press, Cold Spring Harbor, N.Y.

1. A method for detecting tumor cells contained in a biological sample by highlighting the presence of allelic imbalances in at least fifteen, preferably at least thirty chromosomal insertion-deletion markers, said method comprising at least the following steps:
a) amplifying said markers by quantitative multiplex PCR;
b) calculating the ratio R of the height of two peaks corresponding to two alleles of each marker;
c) calculating an overall statistical score relating to the set of markers, obtained by comparing the ratio R with a reference ratio R_{f}; and
d) comparing said score with a fixed threshold of normality.

2. A method according to claim 1, in which the reference ratio R_{f} is a mean of the ratios of the peak heights relating to the two alleles of a marker, said ratios being obtained from at least thirty healthy reference samples.

3. A method according to claim 1 2, in which the overall score is calculated using the formula:
Score=Σdᵢ²=Σ{[LnRᵢ−m(LnR_{f})]÷s(LnR_{f})}²,
in which:
dᵢ represents the statistical distance calculated for a marker i;
Rᵢ designates, for each marker, the ratio of the heights of two peaks, calculated from the biological sample;
R_{f} designates, for each marker, the ratio of the heights of two peaks calculated from a reference sample;
m(LnR_{f}) corresponds to the mean of values of LnR_{f}; and
s(LnR_{f}) corresponds to the standard deviation of values of LnR_{f}.

4. A method according to claim 3, wherein the overall score is distributed in accordance with a chi squared law the number of degrees of freedom of which is equal to the number of tested markers.

5. A method according to claim 4, wherein the fixed threshold of normality beyond which the biological sample is considered to be pathological corresponds to the value of chi squared for a selected value for the risk of the first kind a and on the number of tested markers.

6. A method for detecting tumor cells contained in a biological sample by highlighting allelelic imbalances in at least fifteen, and preferably at least thirty chromosomal insertion-deletion markers, said method comprising at least the following steps:
a) amplifying said markers by quantitative multiplex PCR;
b) calculating the ratio R of the height of two peaks corresponding to two alleles of each markers:
(c) calculating an individual distance dᵢ for each marker i, using the formula:
dᵢ=[LnRᵢ−m(LnR_{f})]÷s(LnR_{f})
in which:
Rᵢ designates, for each marker, the ratio of the heights of two peaks, calculated from the biological sample;
R_{f} designates, for each marker, the ratio of the heights of two peaks calculated from a reference sample;
m(LnR_{f}) corresponds to the mean of values of LnR_{f}; and
s(LnR_{f}) corresponds to the standard deviation of values of LnR_{f}.
(d) comparing the value of the distance dᵢ with respect to the selected confidence interval.

7. A method according to claim 6, wherein the confidence interval is defined by the formula:
m(LnR_{f})±[3×s(LnR_{f})]
in which:
R_{f} designates, for each marker, the ratio of the height of two peaks calculated from a reference sample;
m(LnR_{f}) corresponds to the mean of values of LnR_{f}; and
s(LnR_{f}) corresponds to the standard deviation of values of LnR_{f}.

8. A method according to claim 1, wherein the amplification step a) is carried out in duplicate.

9. A method according to claim 8, in which step b) comprises calculating the mean of two ratios R, each of said ratios corresponding to an amplification reaction carried out in accordance with step a).

10. A method according to claim 1, wherein the detection specificity for tumor cells is determined by the selected value of α.

11. A method according to claim 1, applicable to detecting tumors of the bladder, in which the biological sample is of urinary origin, and the reference samples are of sanguine origin.

12. A method according claim 1, in which at least one marker is selected from markers present in the following table:

| Location | Marker |
|---|---|
| 2q | 919, 1559 |
| 3p | 17, 752 |
| 4p | 1983 |
| 4q | 642 |
| 5q | 714, 739, 787 |
| 6q | 402, 440, 471, 627 |
| 8p | 12 |
| 9p | 18, 116, 476, 558, 1410, 1570, 1998, 2065, |
| | 2086, 2102 |
| 9q | 118, 289, 475, 516, 561, 682, 1370, 1384, |
| | 1583, 1584, 1654, 1921 |
| 10q | 390, 671, 686, 1789 |
| 11p | 1704 |
| 11q | 1982 |
| 13q | 22, 562, 894, 1363, 1698, 1792, 1979 |
| 14q | 1319 |
| 16q | 792, 1187 |
| 17p | 273, 663, 1385, 1657, 1770 |
| 18q | 1697, 1228 |

13. A method according to claim 12, wherein at least one pair of primers for specific amplification used in step a), designated AI to O3, is constituted by primers selected in pairs from sequences SEQ ID Nos 1 to 134, and sequences hybridizable under strict conditions to at least 80% therewith.

14. A sequence for use as a primer for specific amplification of a chromosomal insertion-deletion marker selected from the group formed by sequences SEQ ID Nos 1 to 134, and from sequences hybridizable under strict conditions to at least 80% therewith.

15. A pair of primers for specific amplification of a chromosomal insertion-deletion marker selected from the group formed by sequences taken in pairs from sequences SEQ ID Nos 1 to 134, and sequences hybridizable under strict conditions to at least 80% therewith, said pair being designated AI to 03.

16. A kit for detecting tumor cells contained in a biological sample by highlighting allelic imbalances in chromosomal biallelic insertion-deletion markers, said kit comprising at least one pair of amplification primers selected from pairs of primers in accordance with claim 15.
